(19) United States
(12) Patent Application Publication
(10) Pub. No.: US 2003/0181462 A1
(43) Pub. Date: Sep. 25, 2003

(54) Use of BIBN4096 in combination with other antimigraine drugs for the treatment of migraine

(75) Inventors: Henri Doods, Warthausen (DE); Wolfgang Eberlein, Biberach (DE); Klaus Rudolf, Warthausen (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Pharma KG, Ingelheim (DE)

(21) Appl. No.: 10/218,136

(22) Filed: Aug. 13, 2002

Related U.S. Application Data
(60) Provisional application No. 60/315,321, filed on Aug. 28, 2001.

(30) Foreign Application Priority Data
Aug. 17, 2001 (DE) DE 101 39 410.1

(51) Int. Cl. A61K 31/517; A61K 31/48; A61K 31/405

(52) U.S. Cl. 514/252.17; 514/419; 514/288

(57) ABSTRACT

A method of treatment or prevention of headache, migraine or cluster headaches, which method comprises co-administration of a therapeutically effective amount of the compound 1-[N²-[3,5-dibromo-N-[[4-(3,4-dihydro-2(1H)-oxochinazolin-3-yl)-1-piperidinyl]-carbonyl]-D-tyrosyl]-L-lysyl]-4-(4-pyridinyl)-piperazine[BIBN4096BS] or a physiologically acceptable salt thereof and a therapeutically effective amount of a second active antimigraine drug, particularly sumatriptan, zolmitriptan or dihydroergotamin or a physiologically acceptable salt thereof, as well as to the corresponding pharmaceutical compositions and the preparation thereof.

### CROSS-REFERENCE TO RELATED APPLICATIONS

Benefit of U.S. provisional application Ser. No. 60/315,321, filed Aug. 28, 2001 is hereby claimed. This is a continuation-in-part of Attorney Docket No. 1/1248 filed on Aug. 9, 2002.

### BACKGROUND OF THE INVENTION

### 1. TECHNICAL FIELD

The invention relates to a method for the treatment or prevention of headache, migraine and cluster headaches, which comprises the co-administration of the agent BIBN4096BS and another antimigraine drug.

### 2. BACKGROUND INFORMATION

Migraine is one of the most common neurological disorders, involving periodical attacks of headache and nausea as well as a plethora of other symptoms. Although considerable progress has been made, the pathophysiology of migraine is still not understood. However, several observations point to an involvement of Calcitonin Gene-Related Peptide (CGRP). Migraine headache involves the activation of the trigeminal system and dilatation of cranial vessels. CGRP is localized to neurons in the trigeminal ganglia and CGRP levels are increased during a migraine attack, presumably causing the vasodilation observed. Accordingly, it is conceivable that inhibition of CGRP-evoked dilatation of the cranial vessels may provide a novel treatment for migraine headache.

Widely used antimigraine drugs are the so-called “triptans”, e.g. sumatriptan and zolmitriptan. These compounds elicit their antimigraine effects due to their vasoconstrictive properties and presumably their inhibition of the release of the neuropeptide calcitonin gene related peptide (CGRP) (Ferrari, M. D., Saxena, P. R. (1995), 5-HT₁ receptors in migraine pathophysiology and treatment, Eur. J. Neurology, 2, 5-21; Johnson, K. W., Phebus, L. A., Cohen, M. L. (1998), Serotonin in migraine: Theiroes, animal models and emerging therapies, Progress in Drug Research, Vol. 51, 220-244), the levels of which are assumed to be increased during a migraine attack (Edvinsson, L., Goadsby, P. J. (1994), Neuropeptides in migraine and cluster headache, Cephalgia, 14(5), 320-327). A completely novel approach to treat migraine is the use of CGRP antagonists (Doods, H., Hallermayer, G., Wu, D., Entzeroth, M., Rudolf, K., Engel, W., Eberlein, W. (2000), Pharmacological profile of BIBN4096BS, the first selective small molecule CGRP antagonist, Br. J. Pharmacol., 129, 420-423).

WO 98/11128 discloses modified amino acids having CGRP-antagonistic properties, their use and methods for their preparation as well as their use for the production and purification of antibodies and as labelled compounds in RIA and ELISA assays and as diagnostic or analytic auxiliary agents in neurotransmitter research. In view of their pharmacological properties the modified amino acids are thus suitable for acute and prophylactic treatment of headache, particularly migraine and cluster headaches.

One of the compounds specifically disclosed by WO 98/11128 is 1-[N²-[3,5-dibromo-N-[[4-(3,4-dihydro-2(1H)-oxochinazolin-3-yl)-1-piperidinyl]carbonyl]-D-tyrosyl]-L-lysyl]-4-(4-pyridinyl)-piperazine, which has the following chemical structure:

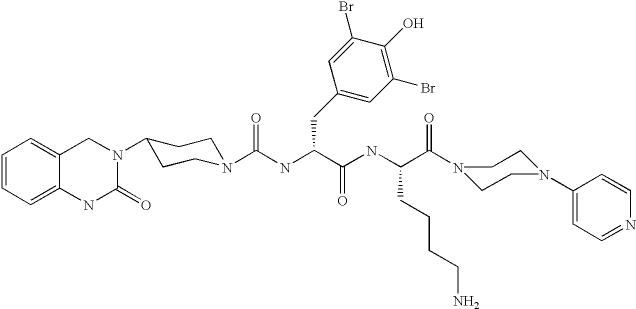

This compound has been identified in the literature by the code name BIBN4096BS. As used herein, BIBN4096BS is intended to refer to and mean 1-[N²-[3,5-dibromo-N-[[4-(3,4-dihydro-2( 1H)-oxochinazolin-3-yl)-1-piperidinyl]carbonyl]-D-tyrosyl]-L-lysyl]-4-(4-pyridinyl)-piperazine.

### BRIEF SUMMARY OF THE INVENTION

Employing a model which is considered to predict the antimigraine effects of drugs, it has been found that the combination of two drugs with a completely different mode of action, namely a 5-HT_{1B/1D} agonist or an ergot alkaloid and the CGRP antagonist BIBN4096BS, yields a significantly and unexpectedly better effect compared to the effect of either drug alone.

### DETAILED DESCRIPTION OF THE INVENTION

As a first aspect the present invention provides a method of treatment or prevention of conditions selected from the group consisting of headache, migraine and cluster headaches, which method comprises co-administration of a therapeutically effective amount of BIBN4096BS or a physiologically acceptable salt thereof and a therapeutically effective amount of another active antimigraine drug, hereinafter referred to as “drug (A)”, which is selected from the group consisting of antiemetics, prokinetics, neuroleptics, antidepressants, neurokinin-antagonists, anti-convulsants, histamine-H1-receptor antagonists, antimuscarinics, β-blockers, α-agonists and α-antagonists, ergot alkaloids, mild analgesics, non-steroidal antiphlogistics, corticosteroids, calcium-antagonists and 5-HT_{1B/1D}-agonists.

A non-steroidal antiphlogistic may be selected from the group consisting of acclofenac, acemetacin, acetylsalicylic acid, azathioprin, celecobix, diclofenac, diflunisal, fenbufen, fenoprofen, flurbiprofen, ibuprofen, indometacin, ketoprofen, leflunomid, lomoxicam, mefenamic acid, meloxicam, naproxen, phenylbutazon, piroxicam, sulfasalazin, zomepirac or the pharmaceutically acceptable salts thereof,

as 5-HT_{1B/1D}-agonists may be used, for example, almotriptan, avitriptan, eletriptan, frovatriptan, naratriptan, rizatriptan, sumatriptan or zolmitriptan or the pharmaceutically acceptable salts thereof and

suitable ergot alkaloids are, for example, ergotamine and dihydroergotamine.

Additional active substances which may be considered for the above-mentioned combinations as drug (A) component include, for example, metoclopramide, domperidon, diphenhydramine, cyclizine, promethazine, chlorpromazine, dexamethasone, flunarizine, dextropropoxyphene, meperidine, propranolol, nadolol, atenolol, clonidine, indoramine, carbamazepine, phenytoin, valproate, amitryptilin, lidocaine or diltiazem.

As a preferred embodiment in the method according to the invention drug (A) is selected from the group consisting of ergot alkaloids and 5-HT_{1B/1D}-agonists, especially preferred are dihydroergotamine, sumatriptan and zolmitriptan, most preferred is sumatriptan or the physiologically acceptable salts thereof.

As a further preferred embodiment in the method according to the invention drug (A) is selected from the group consisting of non-steroidal antiphlogistics, especially preferred is meloxicam or the physiologically acceptable salts thereof.

The dosage for the combined migraine drug (A) is appropriately 1/50 of the lowest dose normally recommended up to 1/1 of the normally recommended dosage, preferably 1/50 to 1/6 and more preferably 1/20 to 1/10, by orally, nasally, subcutaneous or intravenous route. The normally recommended dose for the combined migraine drug (A) should be understood to be the dose disclosed in Rote Liste Win® 2001/I, Editio Cantor Verlag Aulendorf.

According to the invention BIBN4096BS or a physiologically acceptable salt thereof may be administered by intravenous or subcutaneous route in a dosage of 0.0001 to 3 mg/kg of body weight or by oral, nasal or inhalative route in a dosage of 0.1 to 10 mg/kg of body weight once, twice or trice a day, in combination with

sumatriptan or a physiologically acceptable salt thereof which may be administered by oral route in a dosage of 0.03 to 1.43 mg/kg of body weight once, twice or trice a day or by intravenous or subcutaneous route in a dosage of 0.002 to 0.09 mg/kg of body weight once or twice a day or

by rectal route in a dosage of 0.007 to 0.36 mg/kg of body weight once or twice a day or

by nasal route in a dosage of 0.006 to 0.29 mg/kg of body weight once or twice a day or in combination with

Zolmitriptan or a physiologically acceptable salt thereof which may be administered by oral route in a dosage of 0.0007 to 0.036 mg/kg of body weight once or twice a day or in combination with dihydroergotamine or a physiologically acceptable salt thereof which may be administered by oral route in a dosage of 0.001 to 0.07 mg/kg of body weight once or twice a day or

in combination with meloxicam or a physiologically acceptable salt thereof which may be administered by oral route in a dosage of 0.004 to 0.21 mg/kg of body weight once a day.

In a preferred embodiment of the invention BIBN4096BS or a physiologically acceptable salt thereof may be administered by intravenous or subcutaneous route in a dosage of 0.0001 to 3 mg/kg of body weight or by oral, nasal or inhalative route in a dosage of 0.1 to 10 mg/kg of body weight once, twice or trice a day, in combination with

sumatriptan or a physiologically acceptable salt thereof which may be administered by oral route in a dosage of 0.03 to 0.24 mg/kg of body weight once, twice or trice a day or

by intravenous or subcutaneous route in a dosage of 0.002 to 0.015 mg/kg of body weight once or twice a day or

by rectal route in a dosage of 0.007 to 0.06 mg/kg of body weight once or twice a day or

by nasal route in a dosage of 0.006 to 0.048 mg/kg of body weight once or twice a day or in combination with

Zolmitriptan or a physiologically acceptable salt thereof which may be administered by oral route in a dosage of 0.0007 to 0.006 mg/kg of body weight once or twice a day or in combination with dihydroergotamine or a physiologically acceptable salt thereof which may be administered by oral route in a dosage of 0.001 to 0.01 mg/kg of body weight once or twice a day or

in combination with meloxicam or a physiologically acceptable salt thereof which may be administered by oral route in a dosage of 0.004 to 0.036 mg/kg of body weight once a day.

In a more preferred embodiment of the invention BIBN4096BS or a physiologically acceptable salt thereof may be administered by intravenous or subcutaneous route in a dosage of 0.0001 to 3 mg/kg of body weight or by oral, nasal or inhalative route in a dosage of 0.1 to 10 mg/kg of body weight once, twice or trice a day, in combination with

sumatriptan or a physiologically acceptable salt thereof which may be administered by oral route in a dosage of 0.075 to 0.143 mg/kg of body weight once, twice or trice a day or

by intravenous or subcutaneous route in a dosage of 0.005 to 0.009 mg/kg of body weight once or twice a day or

by rectal route in a dosage of 0.0175 to 0.036 mg/kg of body weight once or twice a day or

by nasal route in a dosage of 0.015 to 0.029 mg/kg of body weight once or twice a day or in combination with

Zolmitriptan or a physiologically acceptable salt thereof which may be administered by oral route in a dosage of 0.00175 to 0.0036 mg/kg of body weight once or twice a day or

in combination with dihydroergotamine or a physiologically acceptable salt thereof which may be administered by oral route in a dosage of 0.0025 to 0.007 mg/kg of body weight once or twice a day or

in combination with meloxicam or a physiologically acceptable salt thereof which may be administered by oral route in a dosage of 0.01 to 0.02 mg/kg of body weight once a day.

The present invention provides as a second aspect a pharmaceutical composition for treating or preventing headache, migraine or cluster headaches comprising a therapeutically effective amount of BIBN4096BS or a physiologically acceptable salt thereof and an antimigraine drug (A) selected from the group consisting of sumatriptan, zolmitriptan and dihydroergotamin or a physiologically acceptable salt thereof as a combined preparation for simultaneous or sequential administration.

A pharmaceutical composition according to the invention may comprise a single dosage unit of 0.1 to 10 mg of BIBN4096BS and

a single dosage unit of 1 to 100 mg of sumatriptan or

a single dosage unit of 0.1 to 2.5 mg of zolmitriptan or

a single dosage unit of 0.1 to 5 mg of dihydroergotamin or

a single dosage unit of 7.5 to 15 mg of meloxicam.

All doses or dosage units of a physiologically acceptable salt of an active compound mentioned hereinbefore should be understood as the dose or dosage of the active compound itself.

Furthermore, a pharmaceutical composition according to the invention may be a kit of parts for treating or preventing headache, migraine or cluster headaches, which kit comprises

(a) a first containment containing a pharmaceutical composition comprising a therapeutically effective amount of BIBN4096BS or a physiologically acceptable salt thereof and one or more pharmaceutically acceptable diluents and/or carriers; and

(b) a second containment containing a pharmaceutical composition comprising sumatriptan, zolmitriptan or dihydroergotamin or a physiologically acceptable salt thereof and one or more pharmaceutically acceptable diluents and/or carriers.

A preferred kit of parts comprises sumatriptan in the second containment.

A third aspect of the present invention is the use of BIBN4096BS or a physiologically acceptable salt thereof in combination with another active antimigraine drug (A) for the manufacture of a pharmaceutical composition for treating or preventing headache, migraine or cluster headaches. Drug (A) and preferred embodiments thereof as well as pharmaceutical compositions are mentioned hereinbefore under the first and second aspect of the invention. Most preferred with respect to all aspects of the invention is the combination of BIBN4096BS with sumatriptan or of physiologically acceptable salts thereof.

Several of the drug (A) components mentioned hereinbefore are already on the market, e.g. sumatriptan is sold under the trade name imigran®, zolmitriptan is sold under the trade name ascotop® and dihydroergotamin and the pharmaceutically acceptable salts thereof under the trade name agit®.

BIBN4096BS can be administered using for instance pharmaceutical formulations disclosed in WO 98/11128 or using one of the following pharmaceutical formulations:

capsules for powder inhalation containing 1 mg of active substance,

inhalable solution for nebulisers containing 1 mg of active substance,

propellant gas-operated metering aerosol containing 1 mg of active substance,

nasal spray containing 1 mg of active substance,

tablets containing 20 mg of active substance,

capsules containing 20 mg of active substance,

aqueous solution for nasal application containing 10 mg of active substance,

aqueous solution for nasal application containing 5 mg of active substance, or

suspension for nasal application containing 20 mg of active substance.

### EXAMPLE 1

In order to examine the pharmacological activity of combinations according to the invention the following experiments have been carried out:

Measurement of Facial Skin Blood Flow

Facial skin blood flow was measured by a modified method described by Escott et al. (Escott, K. J., Beattie, D. T., Connor, H. E., Brain, S. D. (1995), Trigeminal ganglion stimulation increases facial skin blood flow in the rat: a major role for calcitonin gene-related peptide, Brain Research, 669(1), 93-99). Fasted male wistar rats (strain CHbb:THOM, 280-320 g) were anaesthetized with sodium pentobarbitone (initially with 60 mg/kg i.p. and maintained throughout the experiment with an intraperitoneal infusion of 30 mg/kg/h through a 23 G needle using a solution of 10 mg/ml). Both sides of the buccal area of the facial skin were shaved and depilated with a commercial depilatory cream (Pilca, Schwarzkopf & Henkel, 40551 Düsseldorf, Germany). The trachea was cannulated and the animals were artificially respired (80 strokes/min) with room air supplemented with oxygen. The body temperature was maintained at 37° C. by an automated heating pad. The left femoral artery and the left femoral vein were cannulated for the continuous measurement of arterial blood pressure and intravenous administration of test compounds, respectively. Neuromuscular blockade was achieved by intravenous administration of pancuronium bromide (1 mg/kg/0.5 ml, 5 min prior to each electrical stimulation). Heart rate was derived from the blood pressure signal. Blood pressure and heart rate were continuously monitored throughout the course of the experiment to assess the level of anaesthesia and to monitor the cardiovascular effects of the drugs used in this study.

The animals were placed in a stereotaxic frame and a longitudinal incision was made in the scalp. A small hole was drilled in the skull (left or right) and a bipolar electrode (Rhodes SNEX-100 supplied by David Kopf Instruments, Tujunga, 91042 California, U.S.A.) was lowered using a micromanipulator, into the trigeminal ganglion (0.32 cm dorsal to bregma, ±0.30 cm lateral from the midline and 0.95 cm below the dural surface). The position of the electrodes in the trigeminal ganglia were checked visually at the end of each experiment following removal of the brain. The trigeminal ganglion was stimulated at 10 Hz, 1 mA, 1 msec for 30 seconds using a stimulator supplied by Hugo Sachs Elektronik (79232 March-Hugstetten, Germany). Microvascular blood flow changes in the facial skin were measured by Laser Doppler flowmetry with a Periflux laser doppler system (PeriFLUX 4001, wave length 780 nM; time constant 3 s, Perimed AB, Järfälla, S-17526, Sweden). Standard laser doppler probes (PROBE 408) were positioned on either side of the face approximately 0.5 cm below the centre of the eye, an area innervated by the maxillary branch (V2) of the trigeminal nerve. Blood flow changes were measured as flux in arbitrary units and expressed as area under the flux curve (mm²) according to Escott et al. (1995).

Experimental Protocol

After 30 min of equilibration, the animals were subjected to three periods of electrical stimulation, separated by a 30 min interval. The first stimulation was used as a control for the subsequent stimulations. Saline, single compound or the combination were administered intravenously 5 min prior to the second stimulation.

The results are given in the following table 1. They show that the improved potency of the combination of 5-HT_{1B/1D} agonists or other antimigraine drugs in general with a CGRP antagonist would allow higher efficacy, would allow lower doses of each compound resulting in similar efficacy with less side effects and the addition of the two mechanisms might result in less headache recurrence.

TABLE 1
Effect of BIBN 4096 BS in combination with other antimigraine
drugs on facial skin vasodilation induced by electrical
trigeminal ganglion stimulation in the rat.

| | | | % inhibition |
|---|---|---|---|
| | % of trigeminus | | compared to |
| treatment | stimulation | n | control value |
| saline (control) | 82.7 ± 4.4 | 11 | — |
| BIBN 4096 BS (0.03 | 60.3 ± 5.1 | 8 | 27.1 |
| mg/kg) | | | |
| Sumatriptan (1.0 mg/kg) | 68.8 ± 6.8 | 7 | 16.8 |
| BIBN 4096 BS + | 26.6 ± 5.4 ^{a} | 6 | 67.8 |
| Sumatriptan | | | |
| (0.03 mg + 1.0 mg)/kg | | | |
| Zolmitriptan (0.1 mg/kg) | 55.6 ± 4.8 | 6 | 32.8 |
| BIBN 4096 BS + | 27.3 ± 6.0 ^{b} | 6 | 67.0 |
| Zolmitriptan | | | |
| (0.03 mg + 0.1 mg)/kg | | | |
| DHE (0.1 mg/kg) | 60.4 ± 4.1 | 6 | 27.0 |
| BIBN 4096 BS + DHE | 20.9 ± 3.1 ^{c} | 6 | 74.7 |
| (0.03 mg + 0.1 mg)/kg | | | |

DHE = Dihydroergotamin
^{a} significant, p < 0.001, compared to Sumatriptan
^{b} significant, p < 0.01, compared to Zolmitriptan
^{c} significant, p < 0.001, compared to DHE

The Examples which follow describe pharmaceutical preparations which contain as active substance BIBN4096BS or a pharmaceutically acceptable salt thereof:

### EXAMPLE 2

Capsules for Powder Inhalation with 1 mg of Active Substance

Composition:

1 capsule for powder inhalation contains:

| | |
|---|---|
| active substance | 1.0 mg |
| lactose | 20.0 mg |
| hard gelatine capsules | 50.0 mg |
| | 71.0 mg |

Method of Preparation:

The active substance is ground to the particle size needed for inhalation. The ground active substance is homogeneously mixed with the lactose. The mixture is packed into hard gelatine capsules.

### EXAMPLE 3

Inhalable Solution for Respimat® with 1 mg of Active Substance

Composition:

| 1 spray contains: | | |
|---|---|---|
| active substance | 1.0 | mg |
| benzalkonium chloride | 0.002 | mg |
| disodium edetate | 0.0075 | mg |
| purified water ad | 15.0 | μl |

Method of Preparation:

The active substance and benzalkonium chloride are dissolved in water and packed in Respimat® cartridges.

### EXAMPLE 4

Inhalable Solution for Nebulisers with 1 mg of Active Substance

Composition:

| 1 vial contains: | | |
|---|---|---|
| active substance | 0.1 | g |
| sodium chloride | 0.18 | g |
| benzalkonium chloride | 0.002 | g |
| purified water ad | 20.0 | ml |

Method of Preparation:

Active substance, sodium chloride and benzalkonium chloride are dissolved in water.

### EXAMPLE 5

Propellant Gas-Operated Metering Aerosol with 1 mg of Active Substance

Composition:

| 1 spray contains: | | |
|---|---|---|
| active substance | 1.0 | mg |
| lecithin | 0.1% | |
| propellant gas ad | 50.0 | μl |

Method of Preparation:

The micronised active substance is homogeneously suspended in the mixture of lecithin and propellant gas. The suspension is transferred into a pressurised container with a metering valve.

### EXAMPLE 6

Nasal Spray with 1 mg of Active Substance

Composition:

| 1 spray jet contains | | |
|---|---|---|
| active substance | 1.0 | mg |
| mannitol | 5.0 | mg |
| disodium edetate | 0.05 | mg |
| ascorbic acid | 1.0 | mg |
| purified water ad | 0.1 | ml |

Method of Preparation:

The active substance and the excipients are dissolved in water and transferred into a suitable container.

### EXAMPLE 7

Injectable Solution with 5 mg of Active Substance per 5 ml

Composition:

| active substance in basic form | 5 | mg |
|---|---|---|
| acid/salt-forming agent in the amount needed | q.s. | |
| to form a neutral salt | | |
| glucose | 250 | mg |
| human serum albumin | 10 | mg |
| glycofurol | 250 | mg |
| water for injections ad | 5 | ml |

Preparation:

Dissolve the glycofurol and glucose in water for injections (WfI); add human serum albumin; add salt-forming agent; dissolve active substance with heating; make up to specified volume with WfI; transfer into ampoules under nitrogen gas.

### EXAMPLE 8

Injectable Solution for Subcutaneous Administration Containing 5 mg of Active Substance per 1 ml

Composition:

| | | |
|---|---|---|
| active substance | 5 | mg |
| glucose | 50 | mg |
| polysorbate 80 = Tween 80 | 2 | mg |
| water for injections ad | 1 | ml |

Preparation:

Dissolve glucose and polysorbate in water for injections; dissolve active substance with heating or using ultrasound; make up to specified volume with WfI; transfer into ampoules under inert gas.

### EXAMPLE 9

Injectable Solution Containing 100 mg of Active Substance per 10 ml

Composition:

| | | |
|---|---|---|
| active substance | 100 | mg |
| monopotassium dihydrogen phosphate = | 12 | mg |
| KH₂PO₄ | | |
| disodium hydrogen phosphate = | 2 | mg |
| Na₂HPO₄.2H₂O | | |
| sodium chloride | 180 | mg |
| human serum albumin | 50 | mg |
| polysorbate 80 | 20 | mg |
| water for injections ad | 10 | ml |

Preparation:

Dissolve polysorbate 80, sodium chloride, monopotassium dihydrogen phosphate and disodium hydrogen phosphate in water for injections (WfI); add human serum albumin; dissolve active substance with heating; make up to specified volume with WfI; transfer into ampoules.

### EXAMPLE 10

Lyophilisate Containing 10 mg of Active Substance

Composition:

| active substance in basic form | 10 | mg |
|---|---|---|
| acid/salt-forming agent in the amount needed | q.s. | |
| to form a neutral salt | | |
| mannitol | 300 | mg |
| water for injections ad | 2 | ml |

Preparation:

Dissolve mannitol in water for injections (WfI); add salt-forming agent; dissolve active substance with heating; make up to specified volume with WfI; transfer into vials; freeze-dry.

Solvent for Lyophilisate:

| | | |
|---|---|---|
| polysorbate 80 = Tween 80 | 20 | mg |
| mannitol | 200 | mg |
| water for injections ad | 10 | ml |

Preparation:

Dissolve polysorbate 80 and mannitol in water for injections (WfI); transfer into ampoules.

### EXAMPLE 11

Lyophilisate Containing 5 mg of Active Substance

Composition:

| | | |
|---|---|---|
| active substance in basic form | 5 | mg |
| polar or nonpolar solvent | 1 | ml |
| (which can be removed | | |
| by freeze-drying) ad | | |

Preparation:

Dissolve active substance in suitable solvent; transfer into vials; freeze-dry.

Solvent for Lyophilisate:

| | | |
|---|---|---|
| polysorbate 80 = Tween 80 | 5 | mg |
| mannitol | 100 | mg |
| water for injections ad | 2 | ml |

Preparation:

Dissolve polysorbate 80 and mannitol in water for injections (WfI); transfer into ampoules.

### EXAMPLE 12

Tablets Containing 20 mg of Active Substance

Composition:

| | |
|---|---|
| active substance | 20 mg |
| lactose | 120 mg |
| maize starch | 40 mg |
| magnesium stearate | 2 mg |
| Povidone K 25 | 18 mg |

Preparation:

Homogeneously mix the active substance, lactose and maize starch; granulate with an aqueous solution of Povidone; mix with magnesium stearate; press in a tablet press; weight of tablet 200 mg.

### EXAMPLE 13

Capsules Containing 20 mg of Active Substance

Composition:

| | |
|---|---|
| active substance | 20 mg |
| maize starch | 80 mg |
| highly dispersed silica | 5 mg |
| magnesium stearate | 2.5 mg |

Preparation:

Homogeneously mix the active substance, maize starch and silica; mix with magnesium stearate; transfer mixture into size 3 hard gelatine capsules in a capsule filling machine.

### EXAMPLE 14

Suppositories Containing 50 mg of Active Substance

Composition:

| | |
|---|---|
| active substance | 50 mg |
| hard fat (Adeps solidus) q.s. ad | 1700 mg |

Preparation:

Melt the hard fat at about 38° C.; homogeneously disperse the ground active substance in the molten hard fat; after cooling to about 35° C., pour into chilled moulds.

### EXAMPLE 15

Aqueous Solution for Nasal Administration Containing 10 mg of Active Substance

Composition:

| | | |
|---|---|---|
| active substance | 10.0 | mg |
| hydrochloric acid in the amount needed to form a neutral salt | 0.01 | mg |
| methyl parahydroxybenzoate (PHB) | | |
| propyl parahydroxybenzoate (PHB) | 0.005 | mg |
| purified water ad | 1.0 | ml |

Preparation:

The active substance is dissolved in purified water; hydrochloric acid is added until the solution is clear; methyl and propyl PHB are added; the solution is made up to the specified volume with purified water; the solution is filtered sterile and transferred into a suitable container.

### EXAMPLE 16

Aqueous Solution for Nasal Administration Containing 5 mg of Active Substance

Composition:

| | | |
|---|---|---|
| active substance | 5 | mg |
| 1,2-propanediol | 300 | mg |
| hydroxyethylcellulose | 5 | mg |
| sorbic acid | 1 | mg |
| purified water ad | 1 | ml |

Preparation:

The active substance is dissolved in 1,2-propanediol; a hydroxyethyl-cellulose solution in purified water containing sorbic acid is prepared and added to the solution of active substance; the solution is filtered sterile and transferred into a suitable container.

### EXAMPLE 17

Aqueous Solution for Intravenous Administration Containing 5 mg of Active Substance

Composition:

| | | |
|---|---|---|
| active substance | 5 | mg |
| 1,2-propanediol | 300 | mg |
| mannitol | 50 | mg |
| water for injections (WfI) ad | 1 | ml |

Preparation:

The active substance is dissolved in 1,2-propanediol; the solution is made up to approximately the specified volume with WfI; the mannitol is added and made up to approximately the specified volume with WfI; the solution is filtered sterile, transferred into individual containers and autoclaved.

### EXAMPLE 18

Liposomal Formulation for Intravenous Injection Containing 7.5 mg of Active Substance

Composition:

| | | |
|---|---|---|
| active substance | 7.5 | mg |
| egg lecithin, e.g. Lipoid E 80 | 100.0 | mg |
| cholesterol | 50.0 | mg |
| glycerol | 50.0 | mg |
| water for injections ad | 1.0 | ml |

Preparation:

The active substance is dissolved in a mixture of lecithin and cholesterol; the solution is added to a mixture of glycerol and WfI and homogenised by high pressure homogenisation or by the Microfluidizer technique; the liposomal formulation obtained is transferred into a suitable container under aseptic conditions.

### EXAMPLE 19

Suspension for Nasal Administration Containing 20 mg of Active Substance

Composition:

| | | |
|---|---|---|
| active substance | 20.0 | mg |
| carboxymethylcellulose (CMC) | 20.0 | mg |
| sodium monohydrogen phosphate/sodium | | |
| dihydrogen phosphate buffer pH 6.8 | q.s. | |
| sodium chloride | 8.0 | mg |
| methyl parahydroxybenzoate | 0.01 | mg |
| propyl parahydroxybenzoate | 0.003 | mg |
| purified water ad | 1.0 | ml |

Preparation:

The active substance is suspended in an aqueous CMC solution; the other ingredients are added successively to the suspension and the suspension is topped up to the specified volume with purified water.

### EXAMPLE 20

Aqueous Solution for Subcutaneous Administration with 10 mg of Active Substance

Composition:

| | | |
|---|---|---|
| active substance | 10.0 | mg |
| sodium monohydrogen phosphate/sodium | | |
| dihydrogen phosphate buffer q.s. ad pH | 7.0 | |
| sodium chloride | 4.0 | mg |
| water for injections ad | 0.5 | ml |

Preparation:

The active substance is dissolved in the phosphate buffer solution, after the addition of the common salt the solution is made up to the specified volume with water. The solution is filtered sterile, transferred into a suitable container and autoclaved.

### EXAMPLE 21

Aqueous Suspension for Subcutaneous Administration Containing 5 mg of Active Substance

Composition:

| | |
|---|---|
| active substance | 5.0 mg |
| polysorbate 80 | 0.5 mg |
| water for injections | 0.5 ml |

Preparation:

The active substance is suspended in the polysorbate 80 solution and comminuted to a particle size of about 1 μm using a suitable dispersing technique (e.g. wet grinding, high pressure homogenisation, microfluidisation, etc.). The suspension is transferred into a corresponding container under aseptic conditions.

1. A method of treatment or prevention of a condition selected from the group consisting of headache, migraine and cluster headaches, which method comprises the co-administration, to a person in need f such treatment, of a therapeutically effective amount of a first agent, which is BIBN4096BS or a physiologically acceptable salt thereof, and a therapeutically effective amount of a second agent, selected from the group consisting of antiemetics, prokinetics, neuroleptics, antidepressants, neurokinin-antagonists, anti-convulsants, histamine-H1-receptor antagonists, antimuscarinics, β-blockers, α-agonists and α-antagonists, ergot alkaloids, mild analgesics, non-steroidal antiphlogistics, corticosteroids, calcium-antagonists and 5-HT_{1B/1D}-agonists.

2. The method according to claim 1, wherein the second agent is selected from the group consisting of ergot alkaloids and 5-HT_{1B/1D}-agonists.

3. The method according to claim 2, wherein the ergot alkaloid is ergotamine or dihydroergotamine or a physiologically acceptable salt thereof and the 5-HT_{1B/1D}-agonist is almotriptan, avitriptan, eletriptan, frovatriptan, naratriptan, rizatriptan, sumatriptan or zolmitriptan or a physiologically acceptable salt thereof.

4. The method according to claim 1, wherein the second agent is sumatriptan, zolmitriptan or dihydroergotamine or a physiologically acceptable salt thereof.

5. The method of claim 4, wherein:
BIBN4096BS or a physiologically acceptable salt thereof is administered by intravenous or subcutaneous route in a dosage of 0.0001 to 3 mg/kg of body weight or by oral, nasal or inhalative route in a dosage of 0.1 to 10 mg/kg of body weight once, twice or trice a day and
sumatriptan or a physiologically acceptable salt thereof is administered by oral route in a dosage of 0.03 to 1.43 mg/kg of body weight once, twice or trice a day or
by intravenous or subcutaneous route in a dosage of 0.002 to 0.09 mg/kg of body weight once or twice a day or
by rectal route in a dosage of 0.007 to 0.36 mg/kg of body weight once or twice a day or
by nasal route in a dosage of 0.006 to 0.29 mg/kg of body weight once or twice a day or
Zolmitriptan or a physiologically acceptable salt thereof is administered by oral route in a dosage of 0.0007 to 0.036 mg/kg of body weight once or twice a day or
dihydroergotamine or a physiologically acceptable salt thereof is administered by oral route in a dosage of 0.001 to 0.07 mg/kg of body weight once or twice a day.

6. A pharmaceutical composition comprising of a first agent, which is BIBN4096BS, or a physiologically acceptable salt thereof, and a second agent, which is selected from the group consisting of sumatriptan, zolmitriptan and dihydroergotamin, or a physiologically acceptable salt thereof, in amounts that are sufficient for treating or preventing headache, migraine or cluster headaches.

7. The pharmaceutical composition of claim 6 comprising a single dosage unit of 0.1 to 10 mg of BIBN4096BS and
a single dosage unit of 1 to 100 mg of sumatriptan or
a single dosage unit of 0.1 to 2.5 mg of zolmitriptan or
a single dosage unit of 0.1 to 5 mg of dihydroergotamin.

8. A kit of parts for treating or preventing headache, migraine or cluster headaches, which kit comprises
(c) a first containment containing a pharmaceutical composition comprising a therapeutically effective amount of BIBN4096BS or a physiologically acceptable salt thereof and one or more pharmaceutically acceptable diluents and/or carriers; and
(d) a second containment containing a pharmaceutical composition comprising sumatriptan, zolmitriptan or dihydroergotamin or a physiologically acceptable salt thereof and one or more pharmaceutically acceptable diluents and/or carriers.

9. The kit of parts according to claim 8, which kit comprises sumatriptan or a physiologically acceptable salt thereof in the second containment.
